(19) United States
(12) Patent Application Publication
(10) Pub. No.: US 2008/0245363 A1
(43) Pub. Date: Oct. 9, 2008

(54) Device and Method For Administration of a Substance to a Mammal by Means of Inhalation

(76) Inventors: Jacob Korevaar, Haarlem (NL); Gerardus Wilhelmus Lugtigheid, Spijkenisse (NL); Ralf Esser, Hennef (DE)

Correspondence Address: GLENN E. KLEPAC, 5818 FORBES AVENUE, SUITE 103, PITTSBURGH, PA, 15217, US

(21) Appl. No.: 10/590,766

(22) PCT Filed: Feb. 24, 2005
(86) PCT No.: PCT/EP2005/002157

§ 371 Date: Jun. 9, 2008

(30) Foreign Application Priority Data
Feb. 24, 2004 (NL) 1025556
May 10, 2004 (NL) 1026154

(51) Int. Cl. A61M 11/00

(52) U.S. Cl. 128/200.23; 128/203.26

(57) ABSTRACT

The present invention relates to a device and a method for administration of a substance to a mammal by means of inhalation. The device according to the invention comprises:
aerosol means, for creating an aerosol,
control means, for manipulating the aerosol in order to thereby control the particle size of the aerosol, wherein
the device is provided with supply means for adding a substance to the aerosol, prior to or upon release of the aerosol from the device.

The device according to the present invention is suitable for pulmonary delivery of substances, such as drugs.

### FIELD OF THE INVENTION

The present invention relates to a device and a method for administration of a substance to a mammal by means of inhalation, wherein the device comprises:
aerosol means, for creating an aerosol,
control means, for manipulating the aerosol in order to thereby control the particle size of the aerosol.

The present invention is specifically suitable for pulmonary delivery of substances, such as drugs.

### BACKGROUND OF THE INVENTION

Traditional drug delivery methods—except injection and infusion—are used primarily with small molecules, such as individual peptides. Pulmonary delivery is already in use for a variety of small-molecule drugs, mainly to treat respiratory disorders. Drugs with respiratory applications include anti-inflammatory agents, bronchodilators and protease inhibitors. Yet, the deep lung is also a favourable environment for non-invasive delivery and absorption of large molecules—as the alveoli (deep lung) provide an extensive air-blood interface allowing large-molecule proteins and peptides access to the body's systemic circulation. Therefore pulmonary drug delivery has the potential to be a much more effective route of administration of macromolecules, with a relatively higher bioavailability than with any other route except injection or infusion. In addition the development of deep lung delivery devices may increase patient acceptance and improve compliance—as an alternative to the invasiveness of injection.

Pulmonary delivery applies to substances with different aggregate conditions: gas, liquid or solid, which may be inhaled both through the nose and the mouth, with the intention to have either a medical or a non-medical effect. Substances for inhalation may be targeted at the body's systemic circulation system, but likewise they may be aimed to have a topical effect from the point of administration onwards, i.e. from the mouth/nose through to the deep lung.

Inhaled liquid and solid substances eventually deposit and are subsequently absorbed, while gaseous substances are taken up through exchange and hence may be partly exhaled. The deposition area of the body comprises the mouth, the nose, the throat, the airway, the bronchi and the alveoli. Gas exchange occurs primarily in the alveoli. The preferred location for deposition is primarily determined by the intended effect and the yield of the substance to be inhaled.

For pulmonary administration of liquid and solid substances to the body, in most cases a device is used that produces a fine particle mist from formulated substances. When administering liquids the mist consists of small moisture particles (aerosol) and in the case of solids a fine powder mist is obtained. The inhalation of such dispersed drugs is most common in the treatment of pulmonary conditions such as asthma, bronchitis, and emphysema. Drug delivery products with respiratory applications include Dry-Powder Inhalers (DPI's), Metered-Dose Inhalers (MDI's), and nebulizers.

During production and inhalation of a fine particle mist from formulated substances, lack of uniformity is an important problem. The particles differ in diameter and usually the particle size distribution is unsymmetrical. As a result the fine particle mist has a mean and median size, a standard deviation and a certain bandwidth. The larger the bandwidth, the wider is the deposition area of the inhaled substances.

Note: mathematically, the most abundant particle size in a mist equals the mean and the median size only in the case of a symmetrical distribution. Some medical specialists however consider the most abundant particle size in a produced mist as the median size even in the case of an unsymmetrical distribution. In this document the mathematical median is used rather than the most abundant particle size.

Investigations have revealed that the particle size of a fine particle mist strongly influences its deposition behaviour. In 1993, the International Committee for Radiation Protection (ICRP) has adapted a new lung model that indicates the deposition rate in different compartments for particles of a specific size. This universally applied lung model was announced in the ICRP 60 report. The old lung model had the following compartments: Naso-Pharynx (NP), Trachea-Bronchi (TB) and Pulmonary (P). The old deposition model considered the aerodynamic behaviour of particles ranging in size from 0,1 to 10 μm only and predicted >40% deposition in the Pulmonary compartment for particles ranging from 0.1 to 0.5 μm, approx. 10% deposition in the compartment TB over the entire range, and >50% deposition in NP for particles over 2 μm.

In the new lung model—also described by A. S. Keverling Buisman in NVS Publication No. 17, pp. 129-134—the compartments have been renamed and regrouped. Naso-Pharynx (NP) has been renamed to Extra-Thoracic (ET), Trachea-Bronchi (TB) has been split into upper Bronchi up to generation 8 (BB) and lower bronchi from generation 9 to 18 (bb). The latter includes part of the old Pulmonary (P) compartment as far as the respiratory bronchi (generation 16 to 18) are concerned. Finally the remaining part of the Pulmonary (P) compartment is renamed to Alveolar-Interstitial (Al). The latter compartment corresponds with the deep lung. In addition to the aerodynamic behaviour of particles in the range 0,1 to 10 μm, the new deposition model also considers thermodynamic behaviour of aerosols in the range 1 to 100 nm. The new deposition model predicts >50% deposition in ET both for particles >2 μm and <2 nm. Optimum deposition (>40%) in AI is predicted in the range 5-50 nm. Deposition in compartment bb is approx. 35% in the range 1-5 nm and <20% for BB for the entire range.

This model implies that as a result of size, an inhaled particle will deposit in a certain location in the trajectory from mouth/nose to the alveoli. In addition the respiratory effort (the flow containing the inhaled fine particle mist in litres per minute) affects the deposition behaviour. A relatively low respiratory effort requires the inhaled particles to be relatively small for optimum deposition in the lower respiratory tract and deep lung. An increased respiratory effort can partly prevent premature deposition of relatively large inhaled particles—e.g. in the upper respiratory tract and lungs.

Individual users of pulmonary delivery devices have their own respiratory profile. Furthermore the conditions of the trajectory mouth/nose to alveoli may strongly vary between users. As a result the deposition behaviour of a substance to be inhaled is difficult to predict. Since the mean particle size of an aerosol to be inhaled is often different from the optimum size for the particular respiratory profile of an individual user in relation to the desired deposition effect of the substance to be inhaled, the expected deposition behaviour strongly deviates from the actual deposition pattern. For that reason control of the deposition behaviour is an important issue.

Existing pulmonary delivery devices produce a fine particle mist from the formulated substance to be inhaled. As a result, the use of such existing devices for administering a substance in many cases leads to inefficient deposition. Existing drug inhalation devices typically deliver only a fraction of the drug to the deep lung, as most of the drug is lost in the delivery device or in the patient's mouth and duroat, due to the fact that the patient must coordinate the breathing manoeuvre with aerosol delivery. Dry-Powder Inhalers and MDI's also fail to provide the deep-lung dosage reproducibility that is necessary for many systemic applications. In addition, therapeutically valuable macromolecules currently cannot be formulated for use in MDI devices, as macromolecule drugs are denatured by the MDI formulating ingredients. A similar problem is associated with drug nebulization, which also tends to inactivate therapeutic macromolecules. In addition, dry-powder devices do not provide the protection needed for the long-term stability of macromolecule formulations. Therefore existing drug inhalation devices such as dry-powder inhalers, metered-dose inhalers and nebulizers are used primarily to deliver drugs to the upper airways for the treatment of topical diseases.

A known device for administration of pharmaceutical preparations is a dry-powder inhaler (DPI). Dry-powder inhalers are breath-actuated devices that use the siphon effect generated by the patient's inhaled air stream to disperse and deliver a drug in fine powder form into the respiratory tract and lungs. When using a dry-powder inhaler a person can breathe in and thereby create a fine power mist from the formulated drug, which is administered to the respiratory tract and lungs. The mist is generated and administered without the need of strict breathing co-ordination that is required for the proper use of a MDI (see below). Dry-powder inhalers do not need propellants and preservatives.

A disadvantage of the use of a dry-powder inhaler is the fact that the functional effectiveness of the apparatus depends on the patient's ability to generate adequate respiratory effort and airflow turbulence for disrupting larger powder formations and producing an aerosol of drug particles of respirable size. Thereby, the siphon that is used to create the mist does not contribute to the reproducibility of the required dose. In addition, dry-powder devices do not provide the protection needed for the long-term stability of macromolecule formulations. Therefore the existing dry-powder inhalers are used primarily to deliver drugs to the upper airways for the treatment of topical diseases.

Despite some functional limitations of DPI's and their higher average price compared to equivalent MDI's, the relative usage of DPI's in the management of COPD patients has expanded rapidly in the past three to four years. Currently, several design versions of DPI's are available in the U.S. including GlaxoSmithKline's Accuhaler™, Diskhaler™, Rotahaler™, Spinhaler™, and Tubuhaler™.

The Accuhaler™ contains a foil strip of 60 blisters, each containing a unit dose of the drug with a lactose carrier. The Diskhaler™ contains a coarse net that creates turbulence to de-aggregate the drug particles. The drug is contained within four or eight foil-blistered discs, allowing multidose administration. The Rotahaler™ is a single-dose device that uses a coarse net to de-aggregate the drug particles and requires reloading with a capsule containing an appropriate drug dose. The Spinhaler™ is a single-dose device that uses a rotor mechanism to expel the drug and requires reloading with a capsule containing an appropriate drug dose. The capsules required in the Rotahaler™ and the Spinhaler™ may be susceptible to moisture. The Turbuhaler™ releases a unit volume of drug into 2 high-resistance, spiral channels, which create a vortex and optimise particle size when the patient's inspiratory flow rate is greater than 30 L/min. This multidose device indicates when 20 doses are left and does not use a propellant, the lack of which reduces coughing and mutes the taste of the drug.

AstraZeneca offers the Pulmicort Tubuhaler™ and the Symbicort Turbuhaler™, a new dry-powder inhaler that offers adjustable dosing, which enables doctors to tailor a patient's treatment with a single inhaler.

The Symbicort Turbuhaler™ is a combination of the budesonide, corticosteroid, and the rapid-onset, long-acting bronchodilator formoterol in a dry-powder inhaler.

Another known device that is used to produce a mist from a formulated drug for administration to a mammal is a so-called metered-dose inhaler (MDI). This type of device is the most widely used delivery device for drug inhalation therapy of COPD.

Metered-dose inhalers use propellants, such as chlorofluorocarbon (CFC's), to release formulated drugs from a pressurised container in order to produce and subsequently deposit a mist of micronized drug particles into the respiratory tract and lungs. The propellant is pressurised and mixed with a fluid containing a formulated drug. When releasing the mixture from the pressurised container an aerosol is formed with micronized particles of typically 1-3 μm. During administration, the particles will travel into the airways as far as halfway the bronchi, using the propellant as a carrier. Thereafter the propellant will evaporate, leaving the remaining particles in the respiratory tract and lung and allowing them to travel deeper into the lung system. The fact that the mixture of propellant and formulated substance particles is fed into the respiratory tract and lungs and the fact that the propellant has to evaporate initially in order to allow the formulated particles to move on, creates a time delay when administering the drug to a patient.

In the MDI-device the container canister is sealed with a special metering valve designed to release a predetermined volume of drug-containing aerosol in each actuation. Within the MDI, the drug is suspended in a propellant with added lubricants and surfactants. Various devices can deliver up to 400 doses; the container's lifetime depends on the volume of drug delivered per actuation.

An advantage of an MDI device, when compared with the above-mentioned DPI, is the fact that the devices are resistant to moisture and relatively cheap.

An important disadvantage of the MDI-device is that fact that an exact co-ordination is required between the actuation of the device and the inhalation. The deposition of particles will depend on the co-ordination of the creation of an aerosol from the formulated drug and the inhalation of a patient.

Lung deposition from a MDI is further affected by the position of the inhaler in relation to the lips, the lung volume at inhalation, the inhaled flow rate and the breath holding of a user after the inhalation (typically for 10 seconds).

Other problems include the lack of a dose counter and the “cold Freon” effect, in which the patient stops inhalation as the aerosol reaches the throat. The low temperature of the mixture entering the body and the reflex of the user not wanting to inhale the cold fluid causes this effect.

In order to improve the operability of the MDI-devices, a breath-actuated MDI was developed to improve the efficiency of drug delivery in patients who have difficulty in coordinating their breathing efforts with the working cycle of a conventional pressurised MDI. A breath-actuated MDI combines a conventional MDI with a spring-driven activation mechanism, which requires priming and is triggered by the patient inhaling at flow rates of 30 L/min or more.

This requirement limits the usability of the device, since many patients, such as COPD patients, will not be able to generate the required flow rate.

Breath-actuated MDI do not require the co-ordination that is necessary with conventional MDI; however, some patients are startled by the release of the spring, which causes glottic closure. This problem may be overcome by using some of the newer MDI, which feature special, quieter activation mechanisms. The clinical efficacy of a breath-actuated MDI device is equivalent to that of a correctly used conventional MDI device in asthmatics and COPD patients.

A further attempt to improve the use of the MDI's is the use of plastic spacers or holding chambers in order to overcome poor co-ordination of actuation by the patient and the cold Freon effect. Spacers are attached to the opening for release and are available in different sizes. Small-volume spacers are available as integral or detachable components of MDI's. Large-volume spacers, which are sold separately and typically replaced every 6 to 12 months, allow the velocity of the aerosol to decrease before inhalation, allowing time for propellant evaporation and reduction in droplet diameter to less than 5 μm, thereby increasing pulmonary drug deposition. With large-volume spacers, high-velocity particles are deflected into the inhaled stream, increasing the efficiency of drug delivery.

An important drawback of the use of spacers, however, is that repeated actuations of the MDI and delayed inhalation from the spacer is associated with up to 50% loss in drug delivery to the respiratory tract and lung. These effects result from both static electricity and the fact that the half-life of the drug aerosol within the spacer is only 10 seconds. Weekly washing, with the spacer left to stand after rinsing, reduces the level of static electricity.

Due to concerns regarding the impact of chlorofluorocarbon (CFC's) on the earth's ozone layer, the Montreal Protocol—a legally binding international agreement—obliges all parties to reduce, then eliminate, all production and use of ozone-depleting substances, particularly CFCs, which have been used as aerosol propellants. As a result, the new CFC-free MDI's are propelled by the more environmentally friendly hydrofluoroalkanes (HFAs). To date, only a few CFC-free MDI models (relying on HFA-based propellants) have been launched in the U.S. However, CFC-free devices are expected to take the place of conventional MDI's in the coming years.

Other companies offering MDI's in the U.S. include Nektar Therapeutics™ and SkyePharma™.

A third type of device for the administration of fluids according to the introduction is a nebulizer. This device produces aerosols from formulated drugs by either passing compressed air rapidly through a liquid containing said drug formulation or by vibrating such a liquid at a high frequency using ultrasound. Both of these methods provide an effective mist for delivering medications. Pneumatic units are considered superior from the standpoint of depth of delivery, as they produce a finer mist that travels deeper into the respiratory tract and lungs, although ultrasonic units are much quieter to operate and do not require a heater.

Despite the fact that the compressor or ultrasound unit represents an equipment investment of at least approximately $125, the actual nebulizer is nearly always purchased as a disposable unit to reduce the risk of cross infection. The exception is with patients who are receiving home healthcare; in some of these cases, the patient may prefer to rely on reusable or semi-disposable nebulizers to reduce costs. Treatment nebulizers are small reservoir, handheld updraft devices used for intermittent delivery of medications. They are used primarily in hospitals and for home-based inimobilised COPD patients. Medication nebulizers are indicated for the delivery of “custom” doses of bronchodilators, corticosteroids, and mucolytics.

AstraZeneca's Pulmicort Respules™ is the first nebulized corticosteroid in the U.S. for use by children as young as 12. After the premixed dose of liquid medicine in the respule is opened, the medicine is poured into a nebulizer, which uses a compressor to aerosolise liquid medication, then delivers it via a facemask or mouthpiece. The NIH recognises the nebulizer as an effective delivery method for infants and young children. Nebulizers are now widely used in the U.S. to deliver nonsteroidal asthma medications. The Pulmicort Respules™ is a preventive measure, not a quick-relief treatment, and is not used to treat asthma attacks.

Beside the apparent disadvantage of the price of the device, an important disadvantage of the present nebulizers is the fact that these devices deliver only a fraction of the drug to the deep lung, as most of the drug is lost in the delivery device or in the patient's mouth and throat.

Investigations have furthermore revealed that both deposition and uptake of an inhaled substance influence the effect and the response time of the administered substance. Therefore the deposition behaviour of the substance to be inhaled must be considered when prescribing a dose and determining the ideal moment of intake. Inefficient deposition—as a result of failure to do so—is an obstacle for pharmaceutical and biomedical companies to ensure optimum use of their substances through inhalation.

For an optimum deposition effect, the deposition behaviour of the substance to be inhaled must be controlled in every possible way. As an example, it may be important to improve the uniformity of the produced mist. In addition, it may be desirable to modify the mean particle size as required, e.g. to adjust it to the respiratory profiles of a generic group of users or that of an individual user. Furthermore it may be desirable to control the concentration of moisture particles, e.g. to moisturise the respiratory tract and lungs of a user.

The United States Patent Application Publication US 2004/0163646 relates to a portable air temperature controlling device for warming air surrounding an aerosolized drug formulation. The heat added to the drug formulation is used to reduce the diameter of aerosol particles produced by an aerosol generation device. The aerosol is formed from a liquid containing a substance, such as a drug. The aerosol particles are reduced in diameter in order to be able to more precisely target the particles to areas of the respiratory tract.

The disadvantages of said portable air temperature controlling device include the fact that an aerosol is produced from a drug formulation, rather than from a pure substance. As a result each particle of the aerosol contains the substance to be administered and is subsequently exposed to the temperature conditions to achieve a reduction in particle size, which limits the use of this device to substances that are able to withstand such temperature conditions and also limits the degree of manipulation and control that can be applied to the aerosol. Furthermore the aerosolised liquid carrier within the formulated drug is completely vaporised, leaving dry powder particles.

### SUMMARY OF THE INVENTION

With reference to the above an object of the present invention is to improve the administration of a substance to a mammal by means of inhalation.

According to a first aspect of the invention this object is achieved in that the invention provides a device for administration of a substance to a mammal by means of inhalation, comprising:
aerosol means, for creating an aerosol,
control means, for manipulating the aerosol in order to thereby control the particle size of the aerosol, wherein
the device is provided with supply means for adding a substance to the aerosol, prior to or upon release of the aerosol from the device.

The device according to the invention may comprise process means, coupled with the control means, provided with storage means, for receiving and processing data relating to a preferred state and condition of the aerosol prior to adding a substance to the aerosol and prior to being administered.

According to a second aspect of the invention a method is provided for the administration of a substance to a mammal by means of inhalation, comprising the steps of:

a) creating an aerosol,

b) manipulating the aerosol by adding or removing energy from the aerosol in order to thereby control the particle size of the particles of the aerosol, and

c) administering the aerosol to the mammal,

wherein the method comprises the step of:

d) adding a substance to the aerosol, prior to the administration of the aerosol to the mammal, in order to administer the substance to the mammal by means of the aerosol.

According to the invention step d) is executed after the completion of step b) and prior to step c). Step b) may be repeated after the completion of step d).

It is possible that the method comprises the steps of:

e) identifying a preferred target area in the respiratory tract and lung system for a substance to be administered to the mammal, and

f) calculating a preferred state and condition for the aerosol.

According to the invention steps e) and f) are executed prior to step b). Step f) may be repeated after the completion of step d).

A result of these measures is the ability to control the conditions of a mixture of an aerosol carrier and a substance added to this aerosol before inhalation, in order to positively influence the deposition behaviour of the substance to be inhaled and the intended effect of the substance.

The aerosol is used as a carrier means for transporting a substance, such as a drug, to the respiratory tract and lungs of a mammal. In a first phase the aerosol is manipulated in order to present optimal characteristics to transport a substance to be administered to the mammal. The aerosol may be manipulated in order to also comply with comfort requirements of the mammal. That means that upon release from the device the loaded aerosol may have a preferred temperature, carrier particle concentration, particle size, uniformity and relative humidity.

In a second phase the substance is added to the aerosol carrier. This has as an advantage that the aerosol can be manipulated without the need of taking care of the stability, integrity or other conditions of the substance. As a further advantage, the substance can be stored in a high concentration without the need of adding a carrier material. The substance does not have to contain a carrier in order to be administered. The substance can in any preferred aggregation be added to the aerosol carrier and be transported to the mammal therewith.

Further preferred embodiments and characteristics of the invention are described in the depending claims.

Further objects, advantages and features of the invention will become apparent upon reading the detailed description of the invention in combination with the drawings.

### BRIEF DESCRIPTION OF THE DRAWINGS

FIG. 1 shows schematically the production of a vapour by means of a fuel cell;

FIG. 2 shows a fuel cell stack;

FIG. 3 shows schematically an embodiment of an inhaler with a fuel cell for creating a vapour, enclosed in a housing;

FIG. 4 shows the inhaler according to FIG. 3 provided with a condenser for creating an aerosol;

FIG. 5 shows the inhaler according to FIG. 4 provided with a dilution chamber, for decreasing the dew point of the aerosol;

FIG. 6 shows the inhaler according to FIG. 5 provided with a mixer, for adding a substance to the aerosol, and

FIG. 7 shows an embodiment of the condensation chamber in the device.

### DETAILED DESCRIPTION OF THE INVENTION

Definitions:

In the present text the wording ‘mammal’ is used. The word ‘mammal’ refers to any human being or animal having a respiratory tract and lung system.

In the present text the wording ‘fluid’ is used. This refers to any liquid, gas, aerosol or the like.

In the text wording is used such as ‘the human body’, ‘a patient’ etc. It is to be understood that the disclosed device and method can be used with the same advantages and effect in the administration of fluids to mammals.

In the text the wordings “aerosol” is used. This refers to a mixture of a gas and moisture particles in that gas, including the moisture in a gas state in said gas.

In the text the wording “aerosol source” is used. This refers to a means for producing an aerosol carrier.

In the text the wording “loaded aerosol” is used. This refers to an aerosol carrier to which a substance has been added.

In the text the wording ‘substance’ is used. The word ‘substance’ refers to any substance, active substance, drug or pharmaceutical formulation, which is suitable to be administered to a mammal by means of inhalation.

In the text the wording ‘relative humidity’ is used. This refers to the ratio between the actual quantity of moisture in a gas state in air (actual humidity) at a certain temperature and the saturation quantity of moisture in a gas state in air (saturation humidity) at that temperature.

In the text the wording ‘dew point’ is used. This refers to the temperature of air at which a certain quantity of moisture in a gas state saturates said air (100% relative humidity).

In the text the wording ‘release(d) from’ is used. The wording ‘release(d) from’ refers to any release, either active or passive, actuated, spontaneous or induced.

Overall Process

In the process of administration of an aerosol according to the present invention, the following steps can be identified:

Step 1: In a first process step it is to be determined what the preferred state and condition of the aerosol used for administering an (active) substance should be during administration thereof. The preferred conditions of the aerosol mainly depend on the substance to be delivered and the preferred deposition effect for that substance. Furthermore, the state and condition of the user can play a role. From the preferred state and condition of the aerosol to be released from the device, an intermediate state and condition of the aerosol prior to adding a substance may be derived.

Step 2: In a second process step an aerosol is created.

Step 3: In a third process step the aerosol is manipulated to adjust the state and condition of the created aerosol in order to e.g. control the uniformity and mean particle size of the aerosol, dependent on the preferred state and condition as established in Step 1.

Step 4: In a fourth step a substance is added to the aerosol.

Step 5: In a fifth step the aerosol is administered to a mammal.

According to a preferred device and method all of the five steps mentioned are required in order to prepare and administer a substance to a mammal. Depending on the substance to be delivered however, it is possible to administer a substance not requiring the use of Step 4 during every inhalation. Furthermore, the sequence of steps may be varied as appropriate, where obviously any manipulation of any aerosol requires the prior creation of that aerosol and likewise any manipulation occurs prior to administration. In addition, the preferred state and condition of the aerosol will de determined prior to finishing the manipulation thereof. It is possible repeat some of the steps and to execute some of the steps simultaneously.

Preferred State and Condition of the Aerosol

The preferred state and condition of the aerosol used for administering an (active) substance will depend on the substance (drug, stimulant or other substance) to be added to the aerosol carrier, the preferred target area of that substance in the respiratory tract (pharynx, lungs, etc.), the object of delivering the substance (treatment, pleasure or other) and on the mammal (age, condition, etc.)

Creation of an Aerosol.

The creation of the aerosol is done by means of an aerosol source that may be located outside the device or inside the device.

The aerosol source may produce moisture particles containing a substance, such as medication, or moisture particles forming an inert carrier, such as water. A combination of substances is also possible. According to the present invention however it is preferred that the substance is added after Step 3 has been completed. In case a starting aerosol is produced containing a first substance, that first substance may have a specific function that is different from the main function of the substance added after completion of Step 3, e.g. the first substance may be a fragrance. Different aerosol sources may be used simultaneously in the device.

The mean particle size and the uniformity of a produced mist of moisture particles may vary between aerosol sources. The mean particle size may range from e.g. sub-nano up to sub-millimetre in diameter. Depending on the intended deposition effect of the substance to be inhaled and the ability to control the state and condition of the aerosol, an inhalation device according to the present invention may be equipped with a specific aerosol source. This way the mist of moisture particles may have a state and condition as required for the following manipulation process. The specific aerosol source may be selected from available methods and devices. As an alternative a newly developed aerosol source may be used.

It is possible to produce an aerosol by passing a compressed gas rapidly through a liquid. Such a pneumatic unit provides a fine mist, with a relatively small particle size. Alternatively the liquid may be vibrated at a high frequency using ultrasound to produce an aerosol.

An other method to produce an aerosol is by pumping a liquid through a heated capillary. The capillary is heated to a constant and relatively high temperature. The liquid entering the capillary is volatilised by the application of heat creating a vapour pressure and causing the vapour to exit the capillary as a vapour jet. The exiting jet entrains the ambient air and rapidly cools, achieving the supersaturated conditions necessary for homogeneous nucleation within a few millimetres of the capillary jet. Aerosol formation is complete within a few centimetres of the tip, yielding a low velocity jet of fine aerosol particles at near ambient temperature. The use of such a mist generator will produce an aerosol with a limited uniformity.

Another method provides in a vibrating membrane, wherein the aerosol is produced by forcing a liquid through the membrane. This method also provides a fine mist, with a relatively small particle size.

According to the invention, a preferred method to produce an aerosol is to use a catalytic burner, such as a fuel cell. According to this method hydrogen and oxygen, such as ambient air are fed to a traditional fuel cell, creating heat, electricity and molecular water (water in gas phase).

The use of a fuel cell has many advantages. The first advantage is the fact that the creation of the aerosol starts with the creation of a gas containing moisture at molecular level. Therefore this particular method offers the possibility to create an aerosol with a much smaller (mean) particle size than with any other existing method. In addition, aerosols formed from said gas will have an extremely uniform particle size. Both bandwidth and standard deviation of the aerosol will be minimal. This uniformity will have the effect that the handling and the farther processing of the aerosol in the device can be predicted and repeated with even greater accuracy than with any other existing method.

A further advantage of the use of a fuel cell is the fact that the generation of the gas will produce electricity, which may be used for the control systems in the device. Since the aerosol is used for e.g. drug delivery purposes, a further advantage is that the created water will be sterile. In addition, a filter may be used to purify the flow—as most delivery devices make use of ambient air. This filter will remove particles, bacteria and/or viruses from the flow.

In order to create an aerosol from the formed gaseous water, the gas will be transported to a condensation chamber, which is a relatively simple technical device. It suffices to have an enclosed space with a controlled temperature.

Similarly a catalytic process may be used to produce a gas. In that case only thermal energy is produced and no electrical energy. A catalytic process may use a liquid fuel, such as methanol instead of hydrogen, wherein a substance may be dissolved or where the substance is coupled with the fuel. During the catalytic conversion the substance is liberated in a predetermined form. In this way the addition of the substance is integrated with the creation of an aerosol. According to the present invention however it is preferred that the substance is added after Step 3 has been completed. As mentioned previously, in case a starting aerosol is produced containing a first substance, that first substance may have a specific function that is different from the main function of the substance added after completion of Step 3, e.g. the first substance may be a fragrance.

As mentioned the aerosol may be formed directly from a liquid containing a substance to be inhaled as well. In that case care must be taken that this substance is able to resist both the conditions arising in the aerosol source e.g. high temperatures needed to volatilise liquids, and the conditions arising during the process of manipulation and control of the aerosol.

Manipulation of the Aerosol

In Step 3 the aerosol is manipulated and controlled, such that a change of state and condition of the aerosol, used for the addition of a substance and used for the administration of that substance, positively influences the deposition behaviour of the substance to be inhaled. For the repeatability of the administration of a substance using an aerosol carrier, it is also important to be able to manipulate or control the state and condition of the aerosol carrier.

The proposed method distinguishes a starting aerosol (input) from a desired aerosol (output). The controlling of both the uniformity and mean particle size of an aerosol, starts with the choice of an appropriate method for creating the aerosol. Depending on the method used for the production thereof, the aerosol will have a typical mean and median particle size and uniformity. The aerosol is manipulated and controlled by adding or extracting energy to/from the aerosol, with the objective to convert moisture from a molecular level (gas) to a liquid state or vice versa. As a result the mean and median particle size and uniformity of the aerosol change.

In order to add energy to or extract energy from an aerosol, both temperature and pressure can be used as control parameters. It is also possible to use a combination of both control parameters. In order to avoid the use of pressurised chambers, in a possible embodiment, it is preferred to use temperature only.

During a condensation process energy is extracted from the aerosol causing the mean particle size to increase and the uniformity to improve. The gas in the starting aerosol becomes saturated and starts to condensate wherein the moisture particles in the aerosol act as condensation nuclei. Since smaller particles exhibit a higher area to volume ratio than larger particles, the smaller particles cool at a higher rate than larger particles. This implies that any condensation will take place at the surface of the smaller particles rather than at the surface of the larger particles. As a result the smaller particles will grow at a higher rate than the larger particles until an equilibrium condition is reached. Consequently the mutual difference in particle size decreases and thus the uniformity of the aerosol. At the same time the mean particle size and median shift to a higher value and the symmetry of the particle size distribution is improved.

During an evaporation process energy is added to the aerosol and the mean particle size decreases. Contrary to condensation, where the number of moisture particles is practically unchanged, during evaporation the number of moisture particles decreases. This time the uniformity improves as well. The smaller particles evaporate at a higher rate than the larger ones. As a result the bandwidth decreases and the particle size distribution becomes more symmetrical.

Thus by adding or extracting energy to/from the aerosol carrier in a controlled way, the uniformity of the aerosol particles and the increase or decrease of the mean particle size of the aerosol can be controlled. As a result a certain median particle size with a certain bandwidth and symmetry can be realised.

In order to determine the specific amount of energy to be added or extracted, the heat content of both the gas and the moisture present in the aerosol carrier are considered. By plotting the heat content and composition of various aerosol and gas mixtures against the temperature, such as in a Mollier type diagram, the amount of energy that must be extracted from the aerosol carrier or added thereto, in order to reach a preferred state and condition can easily be determined. When this specific amount of energy is then actually extracted from the starting aerosol or added thereto, the state and condition of the aerosol carrier change as desired. This is referred to as a regulated condensation process and/or evaporation process.

In order to determine the specific amount of energy that must be added to a starting aerosol or extracted from it, it is also possible to exclusively consider the heat content of the moisture that is present in the aerosol and neglect the heat content of the gas that is present in the aerosol. The result does not necessarily deviate very much from the intention since the yield difference is relatively small. The heat content of a gas is after all orders of magnitude smaller than that of a liquid.

On the other hand it is possible to use the evaporation rate or evaporation yield as reference for certain applications in addition to the heat content. This is done to enable a more accurate manipulation and control of the starting aerosol. For that reason it may be more appropriate to refer to the heat properties of the gas and liquid present in the aerosol carrier.

When the temperature is used as a control parameter to extract a specific amount of energy from an aerosol or add it thereto, the corresponding temperature gradient must be determined. The required temperature gradient is determined on the basis of the heat content of the gas and moisture that is present in the aerosol carrier and the specific amount of energy that must be extracted from the aerosol or added thereto. As a result of the temperature decrease or increase of the aerosol carrier, the desired quantity of moisture will be converted from one aggregate condition to the other. Obviously it is possible to determine the temperature gradient exclusively from the heat content of the moisture that is present in the aerosol carrier and not that of the gas present.

The maximum yield reached during manipulation of the state and condition of an aerosol, is 100%. That means all energy that is extracted from an aerosol or added thereto, is used for the conversion of moisture from the one to the other aggregate condition without changing the degree of saturation of the gas in the aerosol. In practice losses will always occur, even though additional precautions may be taken to reach an optimum yield. The higher the yield, the more accurate the state and condition of an aerosol can be controlled.

### EXAMPLE I

In an inhalation device according to the present invention, an aerosol source is present that produces a starting aerosol. The aerosol source uses existing techniques and produces an unsaturated aerosol. The starting aerosol is subsequently manipulated and controlled, prior to adding a substance to the aerosol, and may be further manipulated and controlled after a substance has been added, such that the loaded aerosol is released from the inhalation device in a preferred state and condition. A certain amount of energy is extracted from the aerosol or added thereto of with the objective to convert a quantity of moisture from the one to the other aggregate condition.

In this example a preferred state and condition of the aerosol is assumed that requires an increase in mean particle size. This implies that moisture at a molecular level (gas) must be converted to moisture in a liquid state. To this effect energy must be extracted from the aerosol, using the temperature as control parameter. In that case the aerosol must be subjected to a certain temperature decrease, in order to convert a specific quantity of moisture from a molecular level (gas) to moisture in a liquid state.

The problem is however, that the gas in the starting aerosol is in an unsaturated condition. As a result a temperature difference exists that must be bridged prior to reaching a saturated gas, which equals a relative humidity of 100% in the aerosol. Only after that, will a further decrease in temperature convert a quantity of moisture at a molecular level (gas) to moisture in a liquid state. This implies that the amount of extracted energy in first instance is obtained from a temperature decrease of the aerosol down to the dew point.

If the starting aerosol prior to manipulation has a relative humidity of e.g. 90%, then the yield is only reduced to a limited extent. This is caused by the relatively small temperature jump that must be realised, prior to reaching the dew point. If the relative humidity is e.g. 40%, the yield reduction is obviously larger. In that case a relatively large temperature jump is required prior to reaching the dew point.

Even if energy is added to the starting aerosol instead of extracted thereof, the yield is reduced. When the relative humidity decreases during evaporation of an aerosol the yield goes down. Adding energy to an unsaturated aerosol results in lower yields than adding energy to a saturated aerosol. For that reason it is preferred that the aerosol source produces a saturated aerosol carrier.

It is obvious that a reduced yield does not contribute to the manipulation and control of the state and condition of an aerosol. In case of a reduced yield, more energy must be extracted from the aerosol or added thereto in order to realise the desired conversion of moisture from the one aggregate condition to the other. The additional energy required for that, is difficult to determine when e.g. the dew point and the relative humidity of a starting aerosol are unknown. In the existing inhalation devices these control parameters are unknown. For that reason the inhalation device and method according to the present invention may use measuring systems that enable a more accurate manipulation and control of the aerosol.

The quantity of gas entering the inhalation device and used to produce a starting aerosol may for instance be measured. In addition the temperature of this gas may be measured. The relative humidity of this gas may also be measured. Furthermore the quantity of moisture that is added to this gas during production of the starting aerosol may be determined. It is also possible to measure the temperature of the moisture particles in the aerosol. By plotting the heat properties of the moisture and gas, that are present in the aerosol to be manipulated, against one or several of the control parameters previously mentioned, the determination of the proper amount of energy required to realise a desired change of state and condition of the aerosol is facilitated to the extent that the manipulation and control can take place with a higher accuracy, the more of the control parameters are known.

An alternative solution is to validate the process of making the starting aerosol. That means from clinical or laboratory investigations the amount of energy actually added to the starting aerosol or extracted thereof, in order to realise the desired conversion of moisture and the desired change of state and condition, is known. The validation is used for making adjustments to the theoretical equations that are applied for manipulation and control.

It is also possible to replace the aerosol source that is used, by an aerosol source that produces a starting aerosol with a relative humidity of 100%. That means, the starting aerosol has reached the dew point. When subsequently energy is extracted from the aerosol or added thereto, the yield will be optimum.

When it is not possible to apply an aerosol source that produces an aerosol with a relative humidity of 100%, or when for instance too few control parameters of the starting aerosol are known, it may be desirable to deploy a condenser in order to bring the starting aerosol in a saturated condition. Because the aerosol is released from the condenser with a known temperature and a relative humidity of 100%, the manipulation and control of the state and condition of the aerosol is facilitated. Thus an optimum starting situation has been created. It is also possible to realise the preferred state and condition of an aerosol directly with the aid of the condenser, such that administration thereof to the body can be the next step.

The modification of the mean particle size and/or the uniformity is not equally important for all applications. The manipulation and control of an aerosol may for instance be used to change an aerosol from an unsaturated condition to a saturated condition. It is also possible that the control means are used to increase or rather decrease the relative humidity of an aerosol. Medications are known for instance that act better at a high than at a low relative humidity and vice versa. The manipulation and control of an aerosol may of course be done with the intention to actually modify the mean particle size and/or the uniformity of the aerosol.

Through the conversion of moisture from the one to the other aggregate condition, a change in the uniformity and the mean particle size is realised. When this modification must result in a specific final value for the uniformity and/or mean particle size, then the uniformity and the mean particle size of the starting aerosol must be known. Based on these starting values the quantity of moisture that must be converted from the one to the other aggregate condition can be determined.

In order to learn the mean particle size of a starting aerosol, prior validation and/or measurement of control parameters may be used. The latter however leads to a more complex inhalation device, whereas for many applications this is not necessary. It is possible to solely consider a state and condition that is expected beforehand. That means, in the past a validation of the production process of the aerosol has taken place and on that basis the uniformity and/or de mean particle size of the starting aerosol can be predicted. In both situations, the starting value is known. Subsequently the quantity of moisture that must be converted from the one to the other aggregate condition can be determined. Next, based on the heat properties of the moisture and gas that are present in the aerosol, the specific amount of energy can then be determined that is required to realise the desired change of state and condition of the aerosol.

The preferred state and condition of the aerosol prior to addition of the substance and/or upon release from the inhalation device, to an extent can be a fixed value. That means, a certain substance with an intended deposition effect is administered and on that basis a certain state and condition of the aerosol are preferred for the administration of that substance. Additional factors that may have a variable influence on the preferred state and condition thereof during the process of making, manipulating and administrating the aerosol are not taken into account.

Considering the preferred state and condition of an aerosol as a fixed value, implies a generalising effect. For some applications this mode of operation will be adequate. When however for a certain application for instance the respiratory profile of the individual user must be taken into account, this cannot be done. Due to the intended deposition effect of a substance to be inhaled, the respiratory effort during inhalation demands a specific state and condition of the aerosol carrying the (active) substance to be inhaled. Therefore the preferred state and condition of the aerosol may differ per user. Furthermore the preferred state and condition of the aerosol may vary during the use of the inhalation device.

The inhalation device and method according to the invention preferably is able to measure in real time in order to determine the preferred state and condition of an aerosol. It is possible to measure for instance the respiratory profile of the user and compare that with a reference lung and use that comparison to control the manipulation of the aerosol. As a result the mean particle size of the aerosol may be adjusted to an optimum value, corresponding with the intended deposition effect of the substance to be inhaled that is carried by the aerosol and the respiratory effort of the user.

### EXAMPLE II

Previously, reference has been made to the use of pressure and/or temperature as control parameters to extract an amount of energy from the aerosol or to add it thereto, with the objective to change the state and condition of the aerosol. Another control parameter that may be used to this effect is the relative humidity. Below an explanation is given with an example.

An inhalation device according to the present invention administers an aerosol to the user based on the flow-through principle. That means, a certain amount of gas is introduced to the device. This is preferably done by using the respiration of the user and/or a supporting mechanism, for instance a ventilator. A certain concentration of moisture particles is added to the flowing gas with the aid of an aerosol source. As a result an aerosol is created. Subsequently the aerosol flows through the inhalation device and the state and condition of the aerosol are manipulated prior to adding a substance and prior to release from the inhalation device. To that effect energy is extracted from the aerosol or added thereto, for which the pressure and/or temperature may be used as control parameters. Preferably the temperature is used as control parameters. Thus no pressure chambers are required and the total volume of the flowing aerosol remains equal, since no gas or other aerosol is added.

It is possible however, that the state and condition of the aerosol are manipulated by adding another gas or another aerosol thereto. This way energy can also be extracted from the aerosol or added thereto. When a gas with a lower relative humidity than that of the aerosol is added, the aerosol will extract energy from the gas. As a result energy is indirectly added to the starting aerosol. When a gas with a higher relative humidity than that of the aerosol is added, the aerosol delivers energy to the gas. This means that energy is indirectly extracted from the aerosol. By controlling this process, it is possible to control the state and condition of the aerosol. Controlling this process means, taking the relative humidity and the volume into account of both the starting aerosol and the gas or aerosol added thereto. By adjusting these control parameters relative to each other, while taking into account the heat properties of the used gas and/or moisture, the state and condition of the starting aerosol are manipulated.

In summary, manipulation and control of the state and condition of a starting aerosol preferably takes place with the aid of a controlled condensation process and/or evaporation process. To that effect the starting aerosol is cooled, heated, diluted or varied in pressure. The previously mentioned control means may be deployed separately or in combination. Thus it is possible for instance to cool initially, heat next and subsequently dilute. Another sequence or combination is also possible.

Upon calculating the preferred state and condition of the aerosol it is further preferred to take into account that the state and condition of the aerosol, and with it the uniformity and mean particle size, may change upon adding a substance and/or upon release from the device and upon entering the body of the mammal.

The additional advantage of applying a temperature gradient to a starting aerosol to reach a saturated condition is, that the temperature of this saturated aerosol is known. Suppose the temperature of the saturated aerosol is 50 degrees centigrade. When the saturated aerosol is subsequently administered to a human, the aerosol will decrease in temperature until the carina is reached where the body maintains a constant temperature of 37 degrees centigrade and a constant relative humidity of 100%. Since the temperature decrease of the saturated aerosol is known, the quantity of moisture that is converted in the trajectory from a molecular level (gas)to a liquid state is known. From this information the growth of the mean particle size, prior to reaching the carina may be deduced.

To prevent the particles of a saturated aerosol from increasing in size as a result of continued condensation, an unsaturated gas, e.g. ambient air may be added, preferably with the same temperature as that of the saturated aerosol released from the condenser. As a result, the degree of saturation of the resulting mixture is reduced, causing the moisture particles in the mixture to partly evaporate. By adjusting the ratio of unsaturated gas and saturated aerosol, the mixture released from the inhalation device will have a predetermined state and condition allowing the fine moisture particles to attain their appropriate size in the trajectory from the device to the preferred target deposition area.

### EXAMPLE III

In an inhalation device according to the invention, a catalytic process is used as the aerosol source. This aerosol source initially produces a gas containing molecules of moisture. This gas is subsequently introduced to a condenser prior to adding the substance to be added. The gas becomes saturated, condenses and is released from the condenser as an aerosol with a certain temperature and a relative humidity of 100%. Subsequently the substance to be added to the aerosol may be added. In order to prevent the added substance to act as condensation nuclei, it may be decided to initially dry the aerosol by dilution prior to adding the substance to be added.

### EXAMPLE IV

An alternative is an inhalation device wherein a fuel cell is used as the aerosol source. The fuel cell produces a gas containing water at a molecular level. The required substance to be added is no part of the produced gas. The gas is led through a condenser prior to adding the substance to be added. The produced gas is released from the condenser as an aerosol with a relative humidity of 100%. Subsequently the substance to be added to the aerosol may be added. In order to prevent the added substance to act as condensation nuclei, it may be decided to initially dry the aerosol by dilution prior to adding the substance to be added.

Once the saturated aerosol is released from the condensation chamber, the aerosol may be diluted by means of a gas, such as ambient air. The dilution of the aerosol means that the condensation process will be inverted. The dilution of the aerosol will decrease the dew point thereof. The dilution of the aerosol, for instance, is achieved by using a gas with essentially the same temperature as the saturated aerosol.

The particle size of the aerosol reaching the opening for release of the device for administration of the aerosol, irrespective of the question whether a substance is added to the aerosol or not, mainly depends on the particle size of the aerosol released from the condensation chamber in combination with the dilution of the aerosol downstream of the condensation chamber.

The combination of condensation and dilution may be used to fine-tune the aerosol in order to obtain an aerosol with the preferred particle size.

In order to manipulate and control the state and condition of the aerosol, the aerosol may be fed to a condensation chamber. The aerosol entering the condenser has a specific state and condition. Usually the aerosol source governs the state and condition of the aerosol. It is possible however that the state and condition of the aerosol are adjusted prior to entering the condenser. To that effect the inhalation device may be provided with the appropriate means.

Thus it is possible that the aerosol entering the condenser is filtered in order to remove fine dust particles or in order to capture some of the moisture particles that are present in the aerosol. Likewise it is possible to partition the aerosol produced by the aerosol source over several condensers. Furthermore several aerosol mixtures may be combined, after which these combined mixtures flow through a single condenser or are partitioned over several condensers. In addition several aerosol mixtures may separately flow through a single condenser or separately or jointly enter several condensers. It is also possible that prior to or upon entering the condenser a substance is added to the aerosol. Furthermore it is possible that the volume of the aerosol is changed prior to entering the condenser; for instance by compression.

The condensation chamber may have the form of an enclosed space, preferably with a first open end to receive the aerosol in the condensation chamber and a second open end to release the aerosol. Because of these features the condensation chamber can be used as a flow-through condensation facility, with a minimal obstruction of the flow of the aerosol towards the opening for release of the administration device. As a result of resistance in the condenser, the flow rate of the aerosol released from the condenser is probably lower than it is when entering the condenser. It is also possible to deliberately increase that flow rate with the aid of supporting means such as an adjustable flow resistance.

In the condensation chamber means are provided to cool the aerosol flowing by. As a result the aerosol is released from the condenser with a lower temperature than it had upon entering. In the condenser cooling may be effected from the outside inwards—that means the walls are cooler than the entering aerosol. It is preferred however to cool from the inside outwards—in that case the walls of the condenser have a temperature that is higher than or equal to the temperature of the aerosol entering and/or continuing through the condenser, while the aerosol flows along and eventually through a cooling element that is placed inside the condensation chamber. This set up has the advantage that no condensation occurs on de walls. A combination of both ways is also possible however.

As mentioned, the aerosol flows along—and preferably through—a cooling element that is placed inside the condensation chamber. As a result the aerosol flowing by will decrease in temperature. The cooling element may be symmetrically placed in the channel; however this is not a prerequisite. Preferably, the walls of the condenser may consist of a material that discourages condensation or may be provided with such a material. In addition measures may be taken to reduce the resistance, such as smooth (plastic) walls—eventually provided with such a material, for instance a coating. The cooling element, along which the aerosol flows, may also consist of material that discourages condensation or be provided with such a material. Condensation on the cooling element is not absolutely required, since a starting aerosol already contains condensation nuclei in the from of small moisture particles.

The temperature may be set to ensure that the aerosol will be fully saturated at the time of release from the condensation chamber. Although in theory the total quantity of moisture in the aerosol released from the condenser should equal the quantity of moisture that the aerosol contained upon entering, in practice the quantity of moisture in the aerosol released from the condenser will probably be lower as a result of ‘losses’. Moisture may for instance be removed by means provided to that effect. This moisture may be transferred to a storage tank in the device or may be discharged outside the device—eventually this moisture may be added to the aerosol source and/or an aerosol entering the condenser. Measures may be taken to capture the moisture particles present in the condenser with a certain size, for instance by moisture separation. Due to a low flow rate it may occur that moisture particles with a certain size are no longer carried and ‘drop out’ of the aerosol.

In order to retrieve the condensation energy, which is released in the condensation chamber a Peltier-element may be used. The energy that can be retrieved by means of said Peltier-element may be used for control systems in the device.

Adding a Substance to the Aerosol

The device according to the invention is adapted to create, manipulate and administer an aerosol, the aerosol being a carrier for the delivery of a substance, such as a drug to a mammal. The substance to be administered is added to the aerosol in a separate step. Thereafter the combination of the aerosol and the substance is further manipulated and controlled or directly administered to a mammal. Optionally the starting aerosol may be produced from a liquid containing another substance. In case a starting aerosol is produced containing another substance, that other substance may have a specific function that is different from the main function of the substance added after completion of Step 3, e.g. the other substance may be a fragrance.

Below different techniques for adding the substance to an aerosol will be described. The techniques relate to the addition of an substance in the state of a gas, liquid or solid to the aerosol. It is to be understood that the methods can be multiplied or can be used in parallel. That means for instance, that a first and a second substance, in the state of a liquid and a substance in the state of a gas may be added to the same aerosol.

In case a substance is added in the state of a medical gas, the doses will be relatively small. In case a large amount of gas is to be delivered to a patient, it will be more practical to use a respiration device.

The medical gas will be present in the device in a canister. The addition of the gas to the aerosol may take place by opening a valve, which closes the opening for release of the canister.

In practice, it is very advantageous to use an aerosol for the administration of a limited amount of gas. In the first place the flow of the aerosol will provide the necessary energy for the transport of the gas to the lung system of a mammal.

The aerosol and the particles in the aerosol will also ensure good mixing of the limited amount of gas with a reasonable amount of ambient air in order to be able to administer the gas in a diluted form.

Since the presence of the aerosol will ensure good mixing, the gas in the canister may have a high concentration of substances, without running the risk of overdosing a certain area in the body of the mammal during the administering of the substance.

A further advantage is the fact that the aerosol, for instance in the form of a water vapour, will also provide the necessary moisture to moisturise the respiratory tract and lung system of a mammal during the administration of the gas.

A still further advantage is that the substance can be added to the aerosol carrier intermittently e.g. once in every eight breath cycles or in a fixed dosage per time unit. In that case the manipulation and control step can be omitted in the breath cycles preceding the addition.

There are several ways to ensure the effective mixing of a substance in the form of a liquid with an aerosol.

According to a first method the liquid containing the substance is pumped through a membrane. The membrane is provided with apertures with a size typically in the range of 0,3-0,7 μm

According to a second method, the liquid containing the substance is put under pressure and allowed to adjoin a membrane provided with apertures. Since the flow of the aerosol adjoins the opposite side of the membrane, the pressurised liquid is allowed to evaporate and as a result a vapour containing the substance is added to the aerosol. The particle size of the particles entering the aerosol flow will depend on the size of the apertures in the membrane. The particle size is relatively small, allowing the particles to evaporate in the aerosol flow.

Due to the effective mixing that can be obtained between the aerosol and the substance added to the aerosol, the substance may be added in small quantities with a high concentration. The advantage thereof is that a small reservoir containing the substance will suffice for a large number of doses. This is possible because of the fact that according to the present invention the substance is not dissolved in its carrier when provided in the administration device, but is added to a separate carrier in the device itself.

Due to the fact that the gas is relatively dry, the passing gas (flow through principle) is ‘hungry’ for moisture. This moisture is formed at the surface of the membrane. At that membrane surface the liquid medication is coming through

According to a third method the substance is dissolved in a propellant, that evaporates instantly upon release, such as CO₂. The propellant and the substance are contained in a canister, closed by means of a valve. Upon opening the valve the propellant and the substance are released and enter the flow of the aerosol. The propellant will instantly evaporate and the substance will be carried towards its destination by means of the aerosol, rather than by the propellant or as a dry powder. This allows the substance to be added to the aerosol carrier at a molecular level. Since the aerosol carrier is manipulated and controlled prior to adding the substance, very small particle sizes can be achieved.

The substance is in the production phase mixed, coupled or bound chemically to an evaporating substance. This evaporating substance will allow the substance to be released from the canister. The evaporating substance will not be used as a carrier to transport and deliver the substance. The evaporation of the substance will allow the substance to be adhered to or mixed with the aerosol. The aerosol will be the carrier transporting the substance to the preferred deposition area.

The evaporating substance will typically evaporate in order to limit the distance of travel of the combination of substance and evaporating substance to less then 50 mm.

In case the propellant is CO₂, the evaporated propellant can be inhaled by a mammal without creating any health risk for the mammal.

In case a substance in the form of a solid is to be added to an aerosol, two cases have to be distinguished.

A first group of solid substances will dissolve in a liquid. Those substances may be added to the aerosol in a way similar to the addition of a liquid to the aerosol.

A second group of solid substances will not dissolve in a liquid. This second group of solid substances may be added to the aerosol in the form of a powder. When the particles of the substance are added to an aerosol carrier, which is not 100% saturated, the particles will not initiate further condensation of the aerosol. The powder will be taken up by the particles in the aerosol and carried by the aerosol. The particle size of the powder will determine the growth of the resulting particle size of the combination of aerosol carrier and powder particles. Since the aerosol carrier is manipulated and controlled prior to adding the powder very small particle sizes can be achieved.

In order to prevent clustering of the powder particles during addition to the aerosol, the particles may be added to the aerosol, using an electrical device in order to provide the particles with a certain electrical charge.

Alternatively the powder particles may be coated to prevent agglomeration. By providing a coating on the powder particles which includes suitable surfactants, the interfacial tension of the moisture particles of an aerosol carrier may be reduced upon adding these coated particles, thus facilitating the uptake.

The level of saturation will be 100% at the level of the carina. The fact that according to the present invention solid substances may be added to a mammal using an aerosol, has the advantage that the inhalation of an aerosol containing a dry powder will be much more comfortable, then the inhalation of a dry powder, for instance using only a gas flow as a carrier.

Administering of the Aerosol to a Mammal

Once the loaded aerosol has reached the opening for release of the administration device, it is ready to be administered to a mammal. Since the aerosol is manipulated to have the preferred particle size for the substance to be delivered, the predictability of the deposition of the substance is greatly improved when compared to prior art devices and methods.

There are several options for the administration of the loaded aerosol to the mammal. The device may be breath-actuated, meaning that the intake of the loaded aerosol will be dependent on the respiratory effort of the mammal. The device may also work with a breath support, meaning that the device will help the mammal with the intake of the flow. The device according to the present invention can also be used in line. It may be used in combination with a mask or a mouthpiece.

Preferably the opening for releasing the loaded aerosol is adapted to be connected to the mouth of a mammal, in order to generate a flow through the device by means of the respiratory effort of the mammal.

According to the present invention the administration of the loaded aerosol may be monitored and managed using a real time control system. This control system requires the use of sensors, control mechanism and process means to fine-tune administration of the substance depending on the specific administration conditions to a preset value for optimum delivery.

The control system must be adapted to fine-tune the amount of substance to be administered and must be able to time the addition of the substance to the aerosol, in a breath cycle.

The flow may be measured directly by means of a sensor or may be deduced using a combination of a flow obstruction and a pressure sensor.

The present invention may be used as a breath operated device. That means a user has to provide a minimum respiratory effort to initiate a flow through the device towards the mouth.

A breath-actuated device may very well be equipped with a fuel cell for the production of the starting aerosol. In this case the user generates a flow, which will be led over a membrane in the fuel cell. The aerosol will then travel via the condensation area towards the opening for release of the device. Prior to being released from the device, a substance may be added to the aerosol carrier.

In this case, the control of the flow through the device is mainly dependent on the momentary respiratory effort of the user.

In case a user is not able to generate any flow, or is only capable of generating a limited flow through the device, additional means may be provided in order to improve the flow through the device towards the opening for release thereof. These additional means may have the form of an appropriate fan or ventilator.

In order to closely monitor the flow in the device-mammal interface, the device is preferably provided with a flow meter. This flow meter is preferably connected to a control mechanism, capable of controlling the additional flow that is to be generated by the ventilator.

During the transfer of the aerosol to the mammal, the respiratory tract and lungs of the mammal will be gradually filled with the incoming aerosol. Since the substance may be added to the aerosol in a separate step, it is possible to select the time of addition of the substance to the aerosol. That means in case the substance is meant to enter the deep lungs, the substance is added to the aerosol at the start of a breath cycle. The substance will be carried to the essentially empty lungs and therefore reach a deeper level of the respiratory tract and lungs then in case the substance was added to the aerosol at the end of a breath cycle. The later the moment the substance is added to the aerosol, the closer to the mouth the deposition area of the substance in the mammal will be.

In order to be able to time the moment of addition of the substance to the aerosol, the device is preferably provided with a combination of a flow meter and a control mechanism, to monitor the flow towards the mammal and to be able to choose the preferred moment for adding the substance to the flow.

Since according to the present invention the substance is added to the aerosol in a separate step, the addition of substance may take place at chosen intervals not necessarily coinciding with every breath cycle. This enables the device to adjust the addition of the substance to the preferences of a specific user.

Thereto the device is preferably provided with means for instance to set a maximum amount of substances to be administered to a user, per time unit. Moreover, the device then preferably comprises means to measure and store the amount of substances added to the aerosol, per time unit. Depending on the use of the device by a specific user, the device then preferably adds substances to the aerosol in order to ensure that the user receives the required dose, without the risk of overdosing.

Prior to adding substances and/or prior to administration to the user, the aerosol is preferably diluted. This dilution may take place by adding ambient air to the flow to be administered to the user.

Interaction of the Different Phases of Creation, Manipulation and Administering of the Aerosol

Above the five individual steps of determination of the preferred particle size and uniformity of the aerosol during administration thereof, the creation of an aerosol carrier, the manipulation of the aerosol the addition of a substance to the aerosol carrier, and the administration of the loaded aerosol to a mammal have been described in detail.

It is to be understood that the different steps 1-5 are interrelated.

For example, in case the amount of aerosol created in a breath-actuated device decreases, due to a decrease in the respiratory effort of the user, the control parameters for the manipulation of the aerosol must be amended in order to ensure an optimum particle size of the aerosol.

The preferred particle size and uniformity of the aerosol depend on the preferred deposition area of a substance in the respiratory tract and lungs of a mammal. The preferred particle size and uniformity also depend on the actual flow of aerosol in a breath cycle.

The actual particle size and uniformity of the aerosol and the flow of aerosol in one breath cycle in combination with the timing of addition of the substance to the aerosol carrier in that breath cycle, determine the resulting deposition area of the substance in the respiratory tract and lungs.

I: The preferred deposition area of the added substance depends on the following parameters:
In case of a malfunction in the body of the mammal, the specific malfunction that is to be treated,
The substance to be delivered to the mammal,
The area in the respiratory tract and lungs to be reached and/or treated with the substance,
The amount of substance to be administered to the mammal,
The age of the mammal.

The indicated parameters are preferably pre-programmed in a device to administer the substance. It is possible to provide the device with process means, such as a computer, which are able to receive and store the specific parameters for a specific use of the device.

It is also possible to add information on the preferred deposition area, and thus the preferred particle size of the aerosol, on the packaging of the substance. This information may be supplied in the form of a Bar Code. Thus when a user inserts a capsule or similar packaging with substance in the delivery device, the device is automatically provided with information on the operational details for the administration of the substance in the capsule to a mammal including the appropriate manipulation and control of the aerosol and the addition of the substance to the aerosol.

II: The actual flow through a device depends on the respiratory effort of a user, in case a breath operated device is used. In a device provided with means such as a ventilator to assist the flow, the actual flow depends both on the user and the additional flow generated by the device. The actual flow is preferably measured upon delivery of the aerosol to the mammal in the fifth step of the process. This information is preferably fed back to the control means in order to (re)calculate the preferred particle size of the aerosol.

In practise, the device is preferably provided with control means to calculate and set a preferred particle size for the aerosol based on an estimated flow. The measurement of the actual flow can then be used to adjust the preset particle size in the device. As an option a minimum number of breath cycles may initially be measured and the actual flow may be used to fine tune the adjusted particle size, prior to the addition of any substance to the aerosol in the Fourth Step, thus ensuring that the substance actually reaches the preferred deposition area.

This means that after a measurement of the flow, the manipulation of the particle size in the device may be changed in order to adapt the particle size to this flow level. Alternatively, the flow may be regulated by using additional flow means in order to obtain the preferred flow level, without the need of changing the particle size of the aerosol. A combination of the two (changing both particle size and flow) could also be used.

The preferred particle size and the required accuracy relating to the bandwidth of the particle size in the aerosol carrier are the main criteria in selecting a technique for the creation of the aerosol carrier.

After the creation of the aerosol carrier the aerosol is manipulated in order to control the particle size of the aerosol. The preferred particle size, as determined in the First Step of the process, determines the degree to which the control parameters regulating the manipulation of the aerosol are varied in order to obtain that preferred particle size.

As previously described, according to the present invention, the manipulation of the aerosol may take place in two steps. In a first step an unsaturated starting aerosol may be saturated, creating a stable starting condition for further manipulation and control of the aerosol with 100% relative humidity and a certain temperature. In a second step the saturated aerosol is preferably diluted with a gas, such as ambient air, in order to prevent the subsequently added substance to act as condensation nuclei. As a result of said dilution the condensation process used in the first step is reversed.

The addition of the substance depends on the following parameters:
The substance to be delivered to the mammal,
The area in the respiratory tract and lungs to be reached and/or treated with the substance,
In case of a malfunctioning in the body of the mammal, the specific malfunctioning that is to be treated with the medication,
The amount of substance to be administered to the mammal,
The age of the mammal,
The psychic condition of the mammal.

The addition of the substance will be interrelated with the actual flow in the device. The addition of the substance is preferably regulated depending on the frequency of the use of the device by a user.

The device may be provided with means to set a maximum amount of substances to be administered to a user per time unit. Moreover, the device preferably comprises means to measure and store the amount of substances added to the aerosol carrier, per time unit. Depending on the use of the device by a specific user, the device can then add substances to the aerosol carrier in order to ensure that the user receives the required dose over a period of time, without the risk of overdosing.

An important effect is the timing of the addition during a breath cycle. The later the moment the substance is added to the aerosol carrier, the closer to the mouth the deposition area of the substance in the mammal will be.

A further aspect of the addition of substances and the control thereof is that the device may be used as a placebo. The user may inhale aerosol carrier at a frequency he prefers, while the device regulates the actual intake of a maximum amount of substance per time unit.

In a similar way, the device may be provided with alarm means to inform a user, in case he has not received sufficient substance per time unit.

The administration of the aerosol will take place from the opening for release of the device. The amount of aerosol and the flow administered from that opening for release depend on the respiratory effort of the user or alternatively on the additional flow generated in the device or on a combination of the two.

The actual flow from the device towards the patient is preferably monitored in order to control the process steps 1-4, as described above and to thereby control the deposition of the substance in the respiratory tract and lungs of the user.

The systems for monitoring the flow from the device to the user may comprise flow sensors. The control system and the sensors may get their energy for operation thereof from a battery in the device or alternatively directly from a fuel cell, in case the latter is present in the device for the production of an aerosol carrier.

Configurations of the invention include bench top (clinical), desktop (residential) and palm-size (handheld) pulmonary delivery devices; however the preferred configuration is a stand-alone personal inhaler (inhalation device).

### DETAILED DESCRIPTION OF THE DRAWINGS

An example of the device is shown in the accompanying drawings. The development of a new drug involves more than the synthesis of a substance that has a particular effect on the body. The developer must also consider how to transport the drug to the appropriate part of the body and, once there, make it available for use.

With advances in drug development, the way in which a drug is introduced into the body is almost as important as the drug itself. Drug concentration must be maintained at a level that provides maximum therapeutic benefit. The goal of drug administration is the achievement of a desired level of drug concentration and therapeutic effectiveness at the receptor site or site of action.

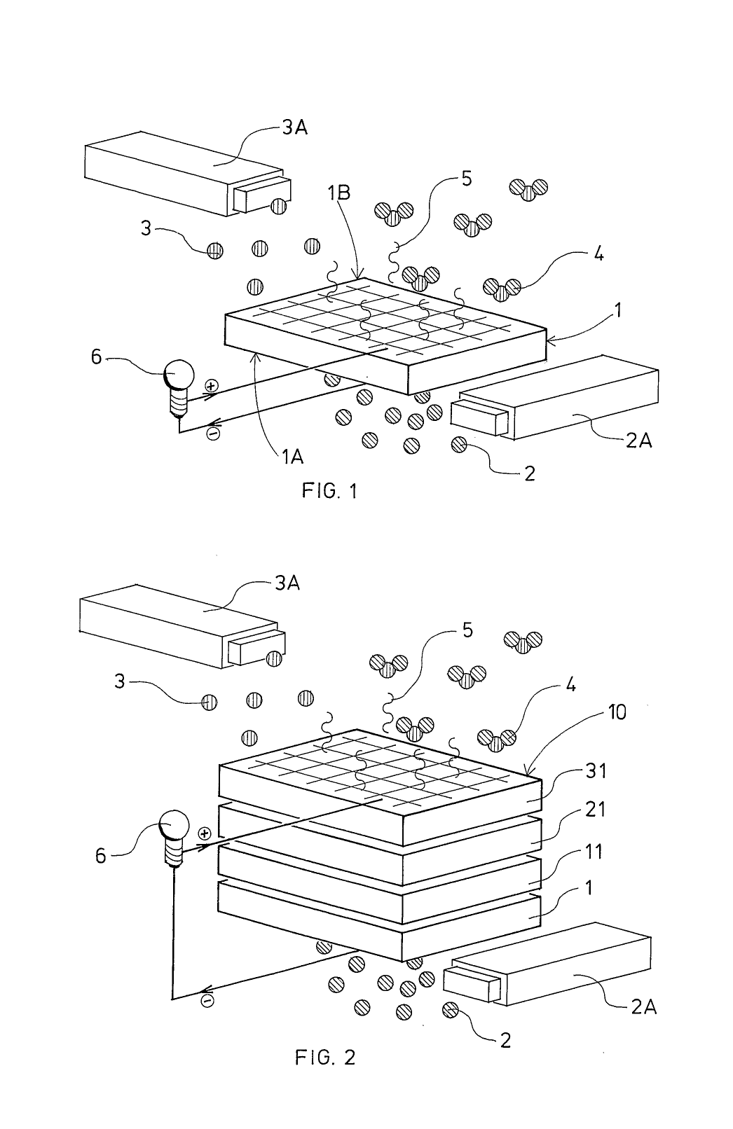

A preferred configuration of the invention, a personal inhaler using a fuel cell is shown in FIG. 1.

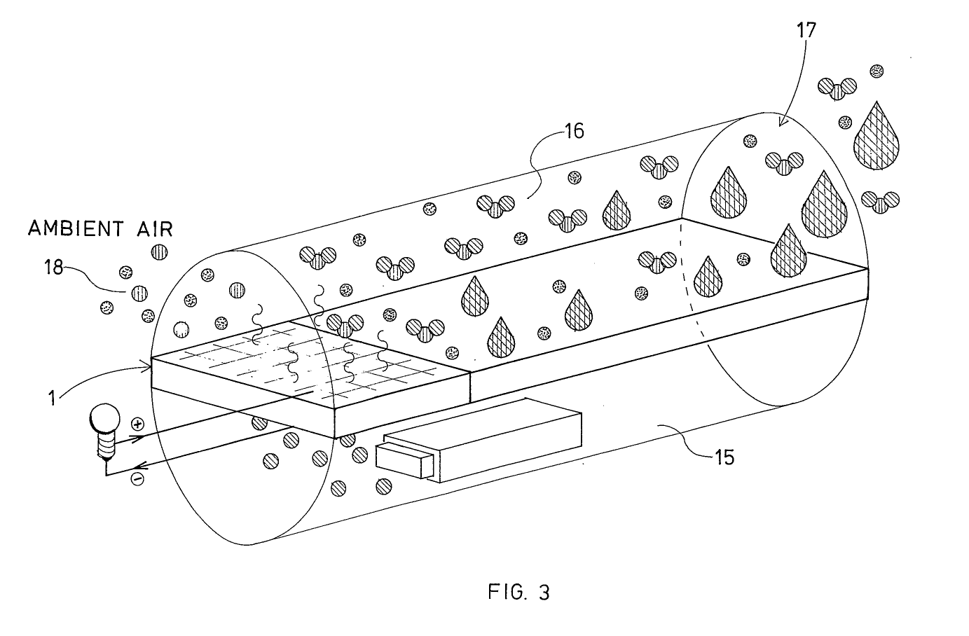

The fuel cell 1 according to FIG. 1 is an electrochemical device that combines hydrogen 2, from a container 2A, and oxygen 3, from a container 3A, to produce water 4, heat 5 and electricity, schematically represented by light bulb 6. Alternatively, the flow of oxygen may be provided by means of ambient air. This is schematically shown in FIG. 3.

As hydrogen 2 flows into the fuel cell's anode 1A and oxygen 3 into the fuel cell's cathode 1B, the fuel cell produces pure water 4 and heat 5. That means that the fuel cell 1 produces a vapour with an elevated temperature.

As shown in FIG. 2, individual fuel cells 1, 11, 21, 31 may be combined into a fuel cell “stack” 10 to increase the total electrical power generated.

The process of generating a warm vapour according to FIGS. 1 and 2, according to the invention is entrapped in an inhaler.

FIG. 3 shows the fuel cell 1 positioned inside an inhaler, schematically represented by cylinder 15. The opening for release 17 for releasing the aerosol is positioned at the right end side of the cylinder 15.

Because of the enclosure 15, the vapour generated in the fuel cell 1 will condense and form a sterile aerosol 16. According to FIG. 3 the required amount of oxygen is provided by the ambient air 18. Alternatively, the oxygen is provided by a container as described with reference to FIG. 1.

Upon travelling through the inhaler, from the fuel cell 1 towards the opening for release 17 the vapour continues to condense causing the particles in the aerosol to increase in size. This is schematically indicated by increasing the size of the represented droplets.

This increase in particle size is undesired, since the particle size of the aerosol determines its stability and the deposition effect.

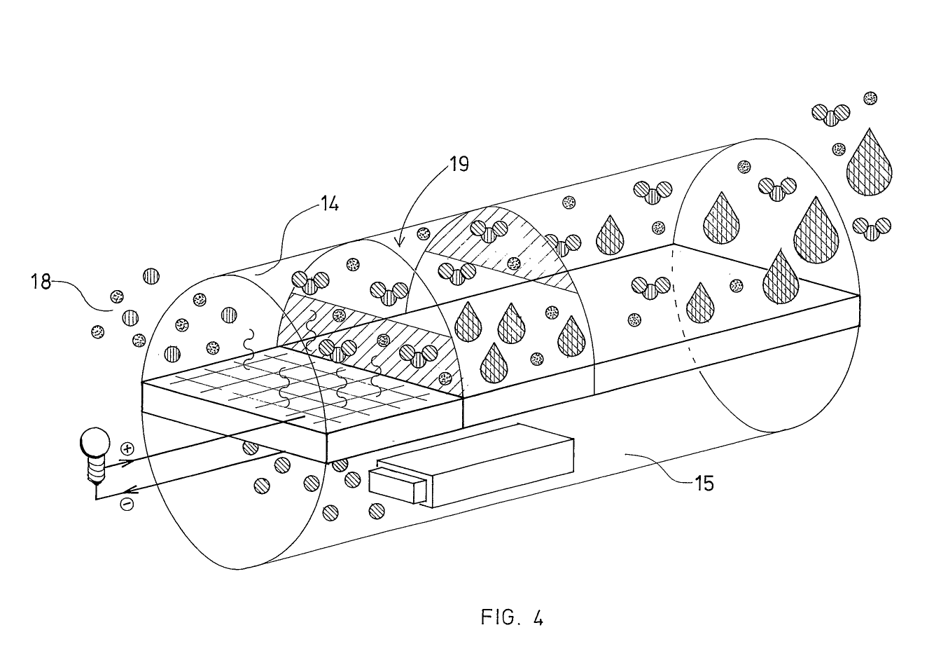

In order to be able to manipulate the particle size in the aerosol, according to the invention, the inhaler 15 is provided with a temperature-controlled condenser 19. This is shown in FIG. 4. In this condenser 19 a saturated mixture is formed. This enables control over the temperature profile of the aerosol and in particular the particle size of the aerosol. The presence of the condenser 19, limits the space wherein the vapour is to be created by means of the fuel cell 1. This enclosed space is referred to as the vapour chamber 14.

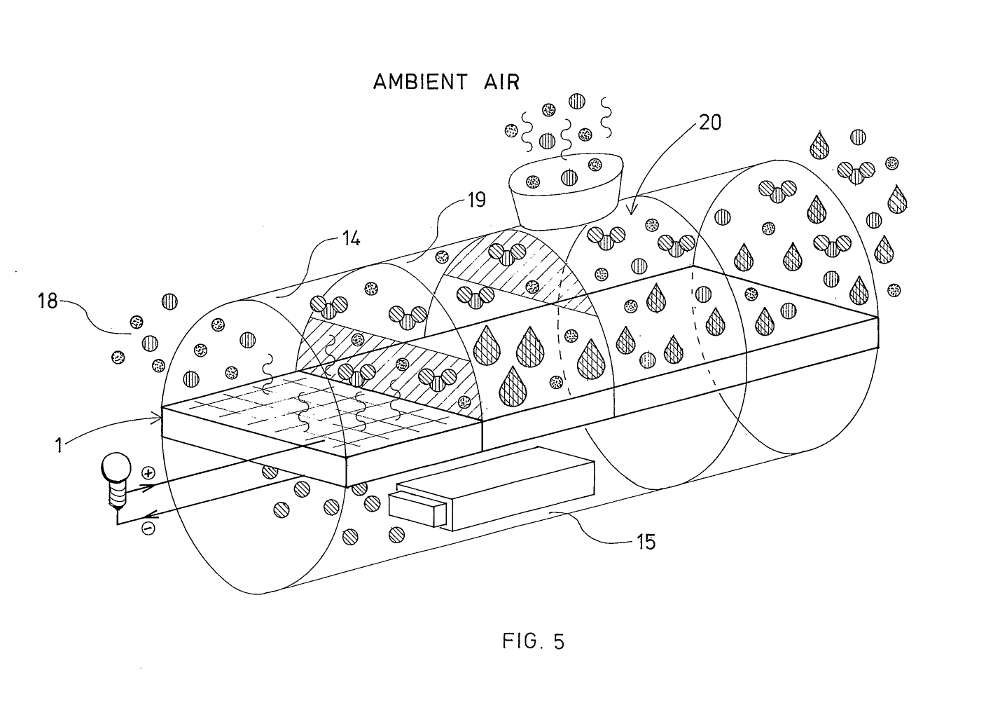

In order to further improve the control over the particle size an unsaturated gas, e.g. ambient air, is added to the aerosol in a dilution chamber 20, as shown in FIG. 5. The unsaturated gas is preferably of the same temperature as the saturated fluid released from the condenser. As a result, the dew point of the mixture is decreased causing the particles in the aerosol to partly evaporate, thereby decreasing the size of the individual particles in the aerosol.

The dew point of the fluid may be further adjusted, to a value below the body temperature. In that case condensation is of the vapour and an increase in particle size is further prevented and the particle size of the aerosol will remain relatively small, even after the aerosol has entered the human cavities. The ratio of unsaturated gas added and the mixture itself determines the new dew point.

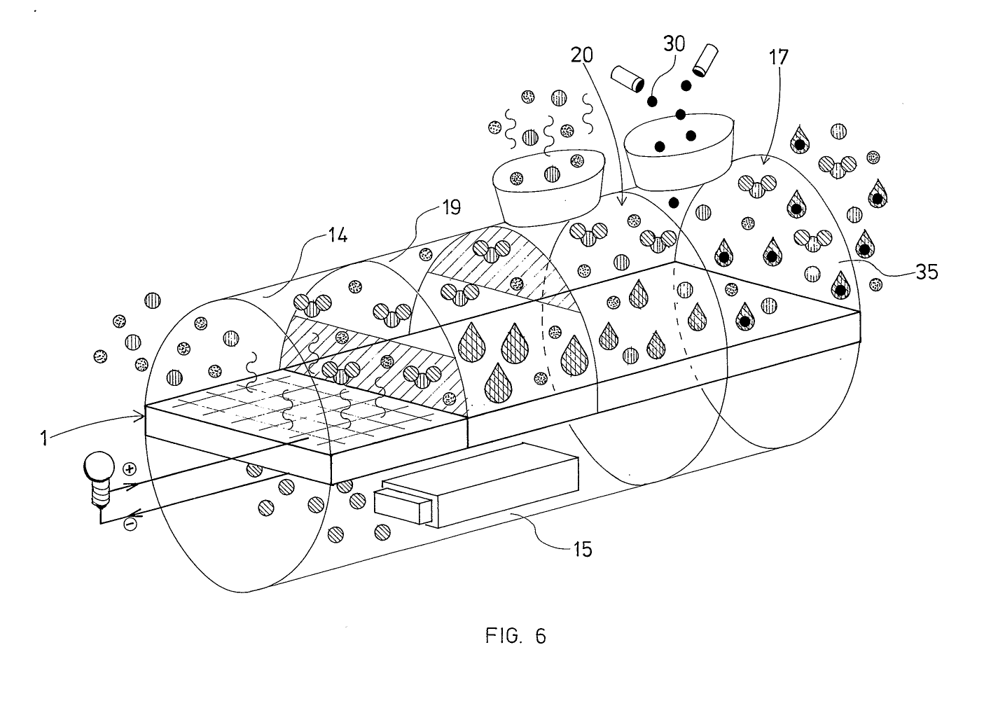

The aerosol that is released from the inhaler is merely a carrier for a substance, such as a drug. The substances 30 are separately added to the aerosol. This is schematically indicated in FIG. 6. The substances 30 are mixed with the aerosol in a mixer 35. The added substances, such as drugs, will combine with the moisture particles in the aerosol, thereby slightly increasing their particle size. The particle size of the particles in the aerosol that are created according to the present invention may be no larger than 20 nanometers.

The substances 30 may be transported to the mixer in the form of a solid, a gas or a substance-aerosol. The aerosol 16 generated in the inhaler will provide the carrier to transport the substances from the mixer 35 towards the human body.

The aerosol 16 generated inside the inhaler 15 will be released from the opening for release 17 with a predetermined temperature. This temperature can for instance be within the range of 20-40 degrees centigrade. This temperature level will eliminate the cold Freon effect. This is a huge advantage over the use of standard MDI devices as described in the introduction.

The aerosol 16 that is released from the inhaler 15 at the opening for release 17 will mainly consist of water droplets. That means that the aerosol, used as the carrier for the substances is a substance which does not have any undesired effect on the human body in general or the lungs in particular. This is a huge advantage over the use of a standard DPI, wherein a dry mist enters the respiratory tract and lungs, causing a grating effect and an itchy feeling within the respiratory tract and lungs.

The shown embodiment is a breath-actuated device. That means that the user himself will have to generate the required respiratory effort to create a flow from the fuel cell 1, via the condenser 19, dilution chamber 20, mixer 35 towards the opening for release 17.

The shown embodiment eliminates the need of having a strict breathing co-ordination for administration of the substances in the respiratory tract and lungs.

It is to be understood that an alternative solution wherein the airflow is generated without the respiratory effort of a user is also feasible.

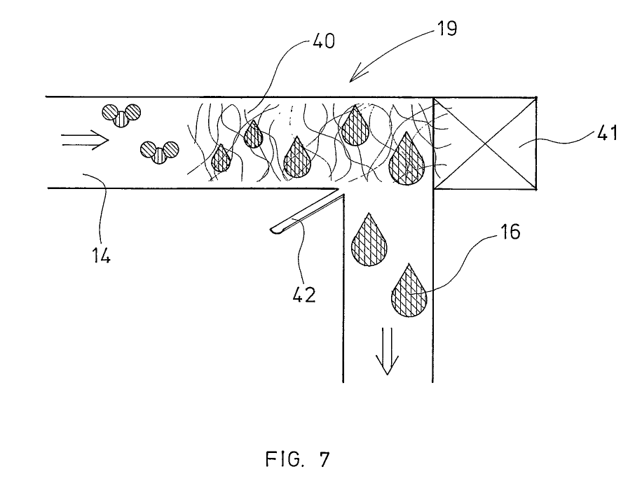

In FIG. 7, schematically, a possible embodiment of a condenser 19 is shown. The aerosol 16 will travel from the gas chamber 14, through the condenser 19, towards the opening for release 17 (not shown) of the device.

The condenser 19 is provided with a heat exchanger 40, preferably comprising an open material, in that the aerosol 16 can flow through the condenser, with a minimal amount of obstruction of the flow by the heat-exchanger.

The heat-exchanger 40 comprises, for instance, metal wool providing a good heat transfer. The wool for instance comprises copper.

The heat-exchanger 40 is coupled with a heating/cooling device 41, in order to regulate the temperature of the heat-exchanger 40.

In the condenser 19 droplets may be formed. These droplets may be collected and led out of the condenser by means of a guide 42. In case the condenser 19 is operated in conjunction with a device for the generation of an aerosol, which uses a liquid to produce the aerosol from, the fluid collected in guide 42 may be fed back to the device for generation of the aerosol.

The conditions in the condenser 19 will be adapted to have an aerosol 16 at the opening for release of the condenser which is 100% saturated. The aerosol released from the condenser will have a stable physical state, in that there is no more condensation or evaporation of the droplets in the aerosol.

As an additional feature the device according to the present invention may be equipped with means to sterilise the device. That means an aerosol is created having a temperature of 100 degrees centigrade, in order to sterilise the device by transporting the aerosol through the device.

With reference to the above it is concluded, that the device and the method as described above provide a cost-effective, clean and sanitary inhalation device. The device is able to proportionally deliver gases, liquids, or solids to the different deposition areas in a human body. The device provides an accurate, controlled and convenient manner of administrating a drug by using an aerosol as a carrier, the aerosol itself comprising a substance, which naturally occurs in the human body. Therefore the aerosol is capable of transporting the administered drug (small and large molecules) to the most effective deposition areas in a human body, without denaturing macromolecules.

The inhaled drug delivery products market is a billion dollar business expected to grow substantially the coming years. The inhaler according to the invention may be developed in clinical, residential and handheld configurations.

The handheld configuration may be provided with a catalytic burner, in particular a fuel cell, which result in a compact and energy self-sufficient personal inhaler. This allows the user to effectively self-administer whatever substance wherever and whenever with a comfort level that will turn inhalation into recreation.

It is to be understood that any other adequate burner can be used without harming the effectiveness of the device.

Since DPI's—Dry-Powder Inhalers—and MDI's—Metered-Dose Inhalers—are known this inhalation device is referred to as D.E.C.I. or DECI—Deposition Effect Controlled Inhaler.

1. Device for administration of a substance to a mammal by means of inhalation, comprising:
aerosol means, for creating an aerosol,
control means, for manipulating the aerosol in order to thereby control the particle size of the aerosol, wherein
the device is provided with supply means for adding a substance to the aerosol, prior to or upon release of the aerosol from the device.

2. Device according to claim 1, wherein the aerosol means comprises a mist generator.

3. Device according to claim 1, wherein the aerosol means comprises a catalytic burner, such as a fuel cell.

4. Device according to one of the preceding claims, wherein the device comprises an aerosol chamber for creating the aerosol in said chamber.

5. Device according to one of the preceding claims, wherein the control means are adapted to add energy to or remove energy from the aerosol in order to thereby control the particle size of the aerosol.

6. Device according to claim 5, wherein the control means comprises a condensation chamber.

7. Device according to claim 6, wherein the condensation chamber has a first open end to receive a flow and a second open end to release a flow.

8. Device according to claims 4 and 6 or 7, wherein the condensation chamber adjoins the aerosol chamber.

9. Device according to any of the claims 5-8, wherein the control means comprises a heat exchanger provided with apertures for allowing the aerosol to pass through the heat exchanger.

10. Device according to any of the claims 5-9, wherein the device comprises a Peltier-element, positioned in the condensation chamber, to retrieve condensation energy.

11. Device according to one of the preceding claims, wherein the control means comprises dilution means for mixing the aerosol with a fluid, such as an unsaturated gas, for thereby decreasing the dew point of the aerosol.

12. Device according to one of the preceding claims, wherein the supply means comprises means for adding a gaseous substance to the aerosol.

13. Device according to claim 12, wherein the supply means comprises a container, such as a canister, for storing a gaseous substance.

14. Device according to one of the preceding claims, wherein the supply means comprises means for adding a liquid substance to the aerosol.

15. Device according to claim 14, wherein the supply means comprises a membrane pump.

16. Device according to one of the preceding claims, wherein the supply means comprises means for adding a solid substance to the aerosol.

17. Device according to claim 14 or 16, wherein the supply means comprises a container for storing a propellant, such as CO₂, and a liquid and/or solid substance.

18. Device according to one of the preceding claims, wherein the device is adapted to be breath actuated.

19. Device according to one of the claims 1-17, wherein the device is provided with means for operating the device with a breath support.

20. Device according to one of the preceding claims, wherein the control means are coupled with process means, provided with storage means, for receiving and processing data relating to a preferred state and condition of the aerosol to be administered.

21. Device according to claim 20 wherein the device is provided with sensor means, coupled with the process means, to measure data relating to the state and condition of the aerosol to be administered.

22. Device according to one of the preceding claims, wherein the supply means are coupled with process means, provided with storage means, for receiving and processing data relating to a preferred timing of the adding of the substance to the aerosol.

23. Device according to claim 22 wherein the device is provided with sensor means, coupled with the process means, to measure data relating to the timing of the adding of the substance to the aerosol.

24. Device according to claim 21 or 23, wherein the sensor means comprise flow measurement means for producing a measure of volume administered.

25. Method for the administration of a substance to a mammal by means of inhalation, comprising the steps of:
a) creating an aerosol,
b) manipulating the aerosol by adding or removing energy from the aerosol in order to thereby control the particle size of the particles of the aerosol, and
c) administering the aerosol to the mammal,
wherein the method comprises the step of:
d) adding a substance to the aerosol, prior to the administration of the aerosol to the mammal, in order to administer the substance to the mammal by means of the aerosol.

26. Method according to claim 25, wherein the method comprises the steps of:
e) prior to step b) identifying a preferred target area in the respiratory tract and lungs for a substance to be administered to the mammal, and
f) calculating a preferred state and condition for the aerosol.

27. Method according to claim 25 or 26, wherein step d) is executed after the completion of step b).

28. Method according to claim 25-27, wherein step b) is repeated after the completion of step d).

29. Method according to claim 25-28, wherein the method comprises the step of:
g) measuring in real time the flow of a first amount of aerosol administered to the mammal, and
h) using the real time measurements in the device-mammal interface in order to control the manipulation of the aerosol in step b) prior to the administration of a second amount of aerosol to the mammal.

30. Method according to claim 25-29, wherein the method comprises the step of:
i) prior to step b) evaluating the heat content of the aerosol in order to thereby determine the specific amount of energy to be added or extracted from the aerosol in order to realise the desired manipulation of the aerosol.

31. Method according to claim 25-30, wherein the manipulation of the aerosol comprises a condensation step, for allowing condensation of at least part of the gaseous phase of the aerosol.

32. Method according to claim 31, wherein the manipulation of the aerosol is adapted to obtain an aerosol with a relative humidity of 100%.

33. Method according to claim 25-32, wherein the manipulation of the aerosol comprises a dilution step, for mixing the aerosol with a fluid, such as an unsaturated gas, for thereby decreasing the dew point of the aerosol.

34. Method according to claim 25-33, wherein the method comprises the step of:
j) using the results of steps e) and g) to calculate a preferred timing for the adding of the substance to the aerosol.

35. Method according to claim 25-34, wherein the method comprises the step of creating in step a) an aerosol containing a first substance and adding in step d) a further substance to said aerosol.
